(19) United States
(12) Patent Application Publication
(10) Pub. No.: US 2020/0243182 A1
(43) Pub. Date: Jul. 30, 2020

(54) SITUATIONAL DYNAMIC BALANCING OF NUTRITION

(71) Applicant: International Business Machines Corporation, Armonk, NY (US)

(72) Inventors: Effron Esseiva, Bowen Island (CA); Romelia H. Flores, Keller, TX (US); Peter G. Finn, Markham (CA); Mark B. Stevens, Austin, TX (US); Shahir A. Daya, North York (CA); Thiago Cesar Rotta, Campinas (BR); Dino Angelo Trevisani, Toronto (CA)

(21) Appl. No.: 16/259,135

(22) Filed: Jan. 28, 2019

(51) Int. Cl. G16H 20/60; A61B 5/145; A61B 5/00; G16H 50/70

(52) U.S. Cl. CPC G16H 20/60; A61B 5/14532; A61B 5/746; G16H 50/70; A61B 5/7275

(57) ABSTRACT

A method, system and computer readable program storage device for managing personalized nutrition. In an embodiment, the method comprises acquiring with a portable device a collection of physical metrics of a user; storing the collection of physical metrics on the portable device; and linking the stored physical metrics with information about the user, pre-stored on a separate storage device, to determine nutrition for the user. The linking is augmented with information obtained over the Internet to identify a place to obtain said nutrition for the user. In an embodiment, the portable device is used to measure a glucose level of the user; the glucose level of the user is compared with thresholds stored in the separate storage device; and based on these thresholds, the portable device issues a warning to the user to additional parties.

### BACKGROUND

This invention generally relates to managing personalized nutrition. More specifically, embodiments of the invention link health parameters acquired with medical wearable or other portable devices, with personal data and preferences to provide a user with nutritional advice.

Medical wearables and handheld devices have become a vital resource for patients to see their current measured health parameters (i.e. pulse, temperature, glucose levels . . . ). Different levels of exercise and stress and food intake constantly alter these parameters, requiring on the fly decisions about what to eat next even in unfamiliar surroundings (i.e. when traveling). This is particularly important for type II diabetics. Inattention or unfamiliarity with available resources may drive the health parameters to dangerous levels, requiring urgent attention. To avoid a critical situation, the user of the handheld device who has just received a threshold notification needs to act and eat the right foods.

One of the problems a user has is to make a complex food decision based on numerous data points. Another problem created by a fast paced life and travel with a medical condition, is the lack of knowledge of the geography and where to find the right food types in either grocery stores or restaurants. Further, as people strive for better health, people need to manage not only their lifestyle preferences such as vegan, halal, kosher, gluten free, lactose free, and personal preferences, but also their health related preferences such as allergy information, intolerances, weight and cholesterol management.

It is often very difficult for a patient to make good on the fly decisions about food. It is also difficult for a health practitioner to step in and assist a patient without being co-located with the patient. Existing solutions require supervision.

### SUMMARY

Embodiments of the invention provide a method, system and computer readable program storage device for managing personalized nutrition. In an embodiment, the method comprises acquiring with a portable device a collection of specified physical metrics of a user; storing the collection of specified physical metrics on the portable device; and linking the stored collection of specified physical metrics with information about the user, pre-stored on a storage device separate from the portable device, to determine nutrition for the user. The method further comprises augmenting the linking with information obtained over the Internet to identify a place, within a defined geographical proximity to the user, to obtain said nutrition for the user.

In an embodiment, the acquiring with a portable device a collection of specified physical metrics of a user includes using the portable device to measure a glucose level of the user; and the linking the stored collection of specified physical metrics with information about the user includes comparing the glucose level of the user with thresholds stored in the separate storage device; and based on said thresholds, the portable device issuing a warning to the user and an additional preset message to additional parties through definable messaging methods.

Embodiments of the invention provide a method and apparatus for dynamically managing and advising nutritional intake to manage glucose intake. One embodiment of the invention provides a method for managing nutritional intake through suggested menus or lists of foods for forthcoming, timely consumption. This method includes defining categories of data. The first category of data is acquired with a portable device to measure blood sugar levels (glucose levels) and is stored on the device. The second category of data is linked information about the individual that is not stored on the device and augmented by a third general category of data on the Web.

One embodiment of the invention includes a method of dynamically managing personalized nutrition to manage glucose intake (i.e., to manage type II diabetes). The method links acquired health parameters with personal data and preferences to advise a patient on what to eat and where; also suggesting food types, grocery stores and restaurants based on geographical location. In another embodiment of the invention, certain persons can be notified of thresholds being crossed.

As mentioned above, it is often very difficult for a person to make good on the fly decisions about food. It is also difficult for a health practitioner to step in and assist a patient without being co-located with the patient. Existing solutions require supervision.

Accordingly, what is needed is a method to dynamically advise the user of the handheld device (or medical wearable) of a balanced nutrition to increase overall health, regardless of where they are, and to provide assistance and notification where needed.

### BRIEF DESCRIPTION OF THE SEVERAL VIEWS OF THE DRAWINGS

FIG. 1 illustrates a method and system in accordance with an embodiment of the invention.

FIG. 2 is a flow chart illustrating an embodiment of the invention.

FIG. 3 shows major components of an embodiment of the invention.

FIG. 4 shows components of a glucose reader that may be used in embodiments of the invention.

FIG. 5 depicts a processing unit that may be used in the practice of the present invention.

### DETAILED DESCRIPTION

In an embodiment, the invention provides a computer implemented method of dynamically managing personalized nutrition. This may be done, as an example, to manage glucose intake (i.e., to manage type II diabetes).

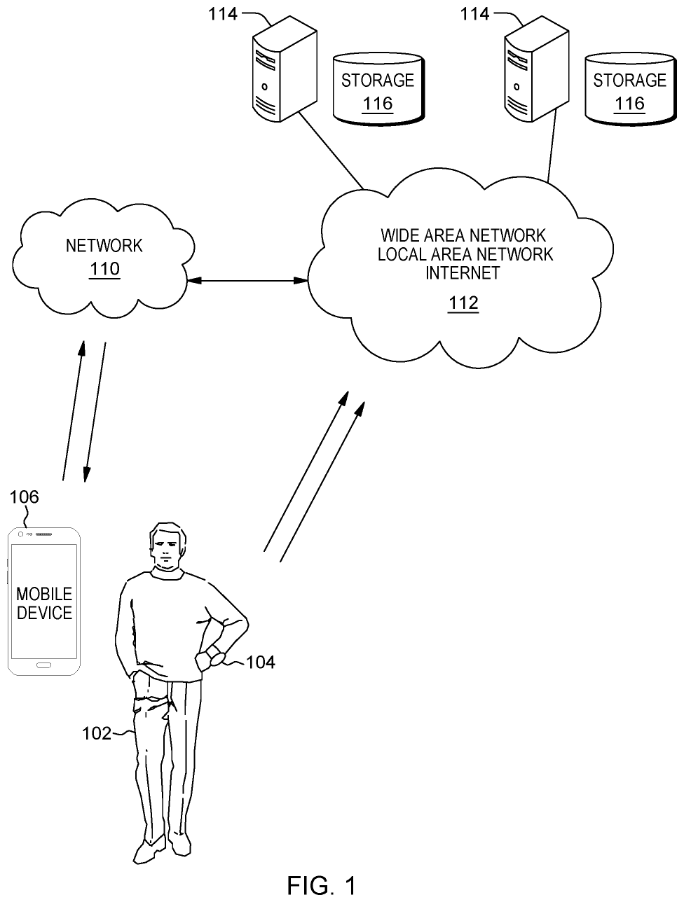

FIG. 1 illustrates a method and system in accordance with an embodiment of the invention. FIG. 1 shows a user 102 wearing a portable sensing device 104, and the user has a mobile computing device 106. FIG. 1 also shows a network 110, a wireless network 112, servers 114 and data storage devices 116. Generally, in an embodiment, sensing device 104 acquires and stores a collection of specified physical metrics of user 102, and the portable device is in communication with network 112 for transmitting data to and receiving data from that network.

In this embodiment, additional information about user 102 is stored on a device separate from portable device 104, and for example, this additional information may be stored on servers 114 or storage devices 116. The collection of specified physical metrics stored on device 104 is linked with the additional information about user 102, pre-stored on the separate storage device, to determine nutrition for the user; and this linking is augmented with information obtained over the Internet to identify a place, within a defined geographical proximity to the user, to obtain that nutrition for the user.

Sensor device 104 is adapted to be placed on or in proximity with the user. In embodiments, sensor device 104 is worn by an individual user on his or her body, for example as part of a garment such as a form fitting shirt, or as part of an arm band or the like. Sensor device 104 includes one or more sensors and a microprocessor. The one or more sensors are adapted to generate signals in response to physiological characteristics of an individual. Proximity as used herein means that the sensors of sensor device 104 are in contact with the user's body, or separated from the user's body by a distance such that the capabilities of the sensors are not impeded.

Sensor device 104 generates data indicative of various physiological parameters of an individual, such as the individual's heart rate, pulse rate, beat-to-beat heart variability, EKG or ECG, respiration rate, skin temperature, core body temperature, heat flow off the body, galvanic skin response or GSR, EMG, EEG, EOG, blood pressure, body fat, hydration level, activity level, oxygen consumption, glucose or blood sugar level, body position, pressure on muscles or bones, and UV radiation exposure and absorption. In certain cases, the data indicative of the various physiological parameters is the signal or signals themselves generated by the one or more sensors and in certain other cases the data is calculated by the microprocessor of the sensor device based on the signal or signals generated by the one or more sensors. Methods for generating data indicative of various physiological parameters and sensors to be used therefor are well known.

Network 110 is enabled to employ any form of computer readable media for communicating information from one electronic device to another. Network 110 can include the Internet, in addition to local area networks (LANs) and wide area networks (WANs).

Servers 114 include virtually any device that may be configured to provide an application service. Such application services, or simply applications, include, but are not limited to, email applications, search applications, video applications, audio applications, graphic applications, social networking applications, text message applications, or the like. In embodiments, servers 114 may operate as web servers. However, servers 114 are not limited to web servers. As illustrated in FIG. 1, each server 114 is connected to a storage device 116, which may be any suitable device for storing data.

Mobile device 106 and network 112 may also be used, in embodiments of the invention. Generally, mobile device 106 may include virtually any portable computing device that is capable of receiving and sending a message over a network such as network 110 and wireless network 112. Such devices include portable devices, such as cellular telephones, smart phones, display pagers, radio frequency (RF) devices, infrared (IR) devices, Personal Digital Assistants (PDAs), handheld computers, laptop computers, wearable computers, tablet computers, integrated devices combining one or more of the preceding devices, and the like. As such, mobile devices typically range widely in terms of capabilities and features.

Those of ordinary skill in the art will appreciate that the architecture and hardware depicted in FIG. 1 may vary. Not all the illustrated components may be required to practice the invention, and variations in the arrangement and type of the components may be made without departing from the spirit or scope of the invention.

In an embodiment, the system of the invention comprises a handheld device (medical wearable) to acquire health details available (i.e., pulse, temperature, glucose levels) of the holder of the handheld device and to store these details. Embodiments of the invention use three categories of data. The first category is data stored on the handheld device. This data includes thresholds, notification preferences and policies to warn family, select persons and doctors, preset warning messages, and geographic location data. A second category of data is stored and managed on a separate system such as a separate server, and this category of data includes policies about likes and dislikes, allergies and lifestyle choices, as well as personal data of the user such as height and weight and health details history. This category also includes a record of food intake, types and estimated calories, and other food and nutritional information about the user.

A third category, or tier, of data is available on the Web that is searched with the first and second categories of data to find information about nearby locations where food is available. This information includes information about restaurants in the current geographical location of the user, a list of restaurants in the location. Menu details may be automatically retrieved from the cloud based on the policies, a list of restaurants may be provided that are suitable for the user, and a personalized menu for the user may be provided. In embodiments of the invention, the information may also include information about grocery stores in the current geographical location of the user, a list of grocery stores in the location. Cloud based policies, and choices of food and meal suggestions may be provided. Information about the aisles where food is available in the grocery stores may be automatically provided.

In embodiments of the invention, the method comprises identifying a collection of electronic metrics and thresholds related to an individual, identifying both specific and generic geographically reachable (in the proximity of the user) providers of nutrition, and linking nutritional values of foods. The method further comprises electronically managing lifestyle preferences (i.e. vegan, halal, kosher, gluten free, etc.), managing personal preferences (i.e. no spinach, no tofu, etc.), as well as health preferences (such as allergy information) and electronically storing communication and notification options based on policies and thresholds. Some of the collection of electronic information is stored on the portable or handheld device and some of the information is stored on a server. The thresholds trigger policy based notifications to select user groups and trigger alarms to the owner of the device to help manage the intake of nutrition. For instance, this may be done for controlling glucose levels. Also, the intake of nutrition may be recorded.

In embodiments, the hand held device compares acquired glucose levels with thresholds, and reads the preferences for the individual and the notification thresholds.

In embodiments, the hand held device issues a warning to the user of the device, and sends an additional preset message based on the threshold to additional parties through definable messaging methods.

In embodiments, the user of the handheld device selects a connection of the glucose levels and thresholds to the preferences and personal data, such as the weight of the person and a calculation is performed to an intake of certain types of food (i.e. an apple, one carrot, 50 g of almonds) from the second category of data.

In embodiments, the user of the handheld device selects a connection of the glucose levels and thresholds as well as the geographic location of the handheld device to the preferences in the second category of data, and uses the retrieved preferences of the user of the handheld and a suggested type of food to search for providers of equivalent nutrition in the geographic proximity of the user.

In embodiments, the user of the handheld device selects a connection of the preferences in the second category of data with searched data in the third category of data from the Web to suggest restaurants that offer the nutrition or food preferences of the user.

In embodiments, the user of the handheld device selects a connection of the preferences in the second category of data with searched data in the third category of data from the Web to show supermarkets that offer the nutrition or food preferences of the user, and to show in what aisles of the stores those preferences are located (if this information is available).

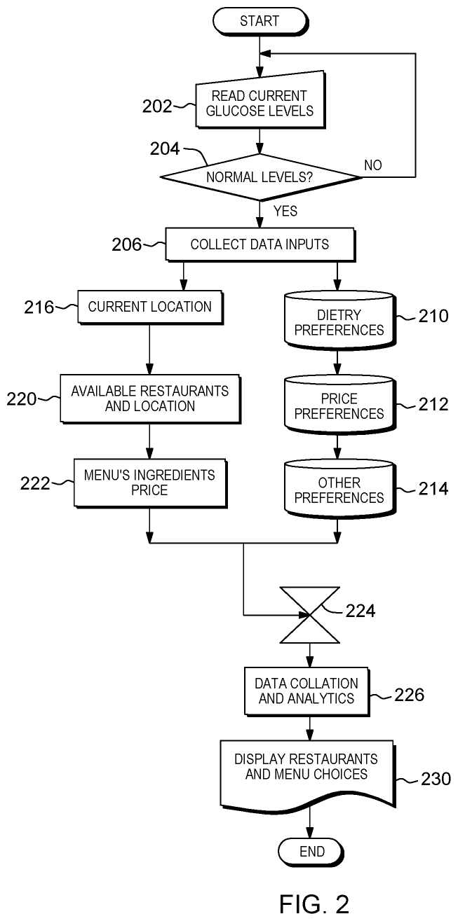

FIG. 2 shows a flowchart illustrating an embodiment of the invention. In this embodiment, the method is used to help a person monitor and maintain their glucose level. At 202, the user's current glucose levels are read; and at 204, the method determines whether these levels are normal. This may be done, for instance, by comparing the read levels to stored data. If the read levels are normal levels, the method returns to 202. As long as the user's glucose levels are normal, steps 202 and 204 are repeated.

If the user's glucose levels become outside the normal levels, the method proceeds to step 206 and various data items are collected. A first group of data items are user preferences, and these include dietary preferences 210 and price preferences 212. Other user preferences 214 may also be collected. The user preferences, in embodiments of the invention, are obtained from server. Additional data that are obtained include the current location 216 of the user, which can be obtained from the wearable device, available restaurants and their locations 220, and the ingredients and prices of items on the menus 222 of the available restaurants. The information about the restaurants, their menus, and the ingredients of the menu items can be obtained from the Web via the Internet.

The data can be filtered at 224, and data collation and analytics are performed at 226. After the data collation and analytics, selected restaurants and menu choices are displayed 230 to the user, and then the method ends. The selected restaurants and menu items can be displayed to the user on the wearable device. This information may also be displayed on the user's cell phone.

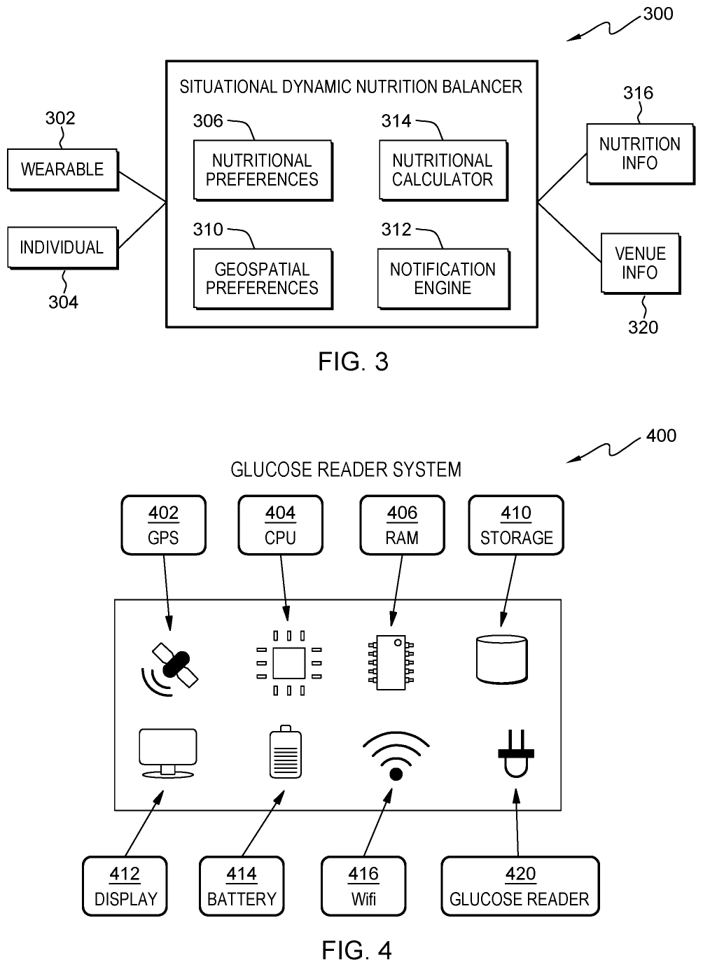

FIG. 3 depicts major components of an embodiment of the invention, referred to as the Situational Dynamic Nutrition Balancer (SDNB) 300. These components include Wearable device 302 which is used by an individual user 304 to feed real-time nutritional or user information into the SDNB in an automated fashion. This information may be received by a medical team or other interested party that receives nutritional status information for the individual user. Nutritional Preferences 306 represents an interface utilized to update nutritional information preferences for the user. This interface enables setting of dietary/food preferences, nutritional levels targeted, allergies, etc. In embodiments of the invention, this interface also tracks associated interested parties for notification. Geo-spatial Preferences 310 represents an interface utilized to track where an individual might be located based on GPS information of their wearable or other device. Based on this information as well as nutritional calculator information and external venue information, this component provides notifications to the Notification Engine 312 with recommended venues for an individual to consider.

Nutritional Calculator 314 is an analytics engine that leverages nutritional information from the web, nutritional preferences of the user, and actual real-time information to calculate appropriate nutritional calculations/recommendations for the user. The Notification Engine is used to push information to a particular individual user for their consideration. Recommendations are based on geo-spatial information. In embodiments, the nutritional information is calculated in real-time. Nutrition Information 316 includes information available in the Internet and provides accurate nutritional information. Venue Info includes information available in the internet about restaurants, stores, etc. and their nutritional ingredients as well as their locations.

In embodiments of the invention, the user provides consent for the collection and use of their data. In embodiments, during a Sign-Up process, the user is asked for consent to store the personal data in a one-step-consent that explains the reason parameters are collected. The personal data are stored with a strong encryption algorithm and acknowledged with a fingerprint, and the personal data are never sent in clear text. In embodiments, the system uses secure communications through SSL and HTTPS, and all system interactions are logged for traceability and auditing purposes. Consent can be withdrawn the same way as consent was given; and, when consent is withdrawn, all data is “forgotten” with a one-step-forget. In embodiments, privacy settings are checked before every interaction and execution of algorithms. During the forget step, i.e. when consent to use private data is revoked, all data related to this user are deleted from the servers and the data are not able to be recovered; interactions and algorithms are no longer possible for this user until new consent is given.

As discussed above, embodiments of the invention may be used to manage glucose intake, and FIG. 4 shows a glucose reader system 400 that may be used in embodiments of the invention. System 400 comprises GPS 402, which is used to identify the location of system 400, CPU 404 that performs the desired computation or processing for system 400, RAM 406 that stores data for system 400, and data storage 410 that stores additional data for the glucose reader system. System 400 also comprises a display unit 412 for providing a visual display of selected data or information, a battery 414 for providing power to the components of the system 400, a wifi component 416 for connecting the system wirelessly to a communications or computer network such as the Internet, and glucose reader 420 for measuring the glucose level of a user. Any suitable components may be used in the glucose reader system 400, and suitable components are well known in the art. Also, the components of the glucose reader system 400 may be connected together and operated in any suitable way.

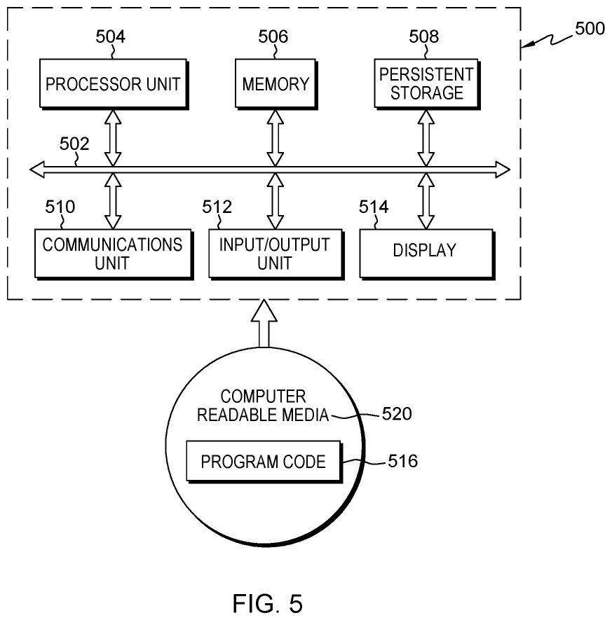

With reference to FIG. 5, a block diagram of a data processing system 500 is shown. Data processing system 500 is an example of a processing unit that may be used in, or with the system of FIG. 1. Data processing system 500 may also be used in or with the Situational Dynamic Balancer 300 of FIG. 3, or in or with glucose reader system 400 of FIG. 4.

In this illustrative example, data processing system 500 includes communications fabric 902, which provides communications between processor unit 504, memory 506, persistent storage 508, communications unit 510, input/output (I/O) unit 512, and display 514.

Processor unit 504 serves to execute instructions for software that may be loaded into memory 506. Processor unit 504 may be a set of one or more processors or may be a multi-processor core, depending on the particular implementation. Memory 506 and persistent storage 508 are examples of storage devices. Memory 506, in these examples, may be a random access memory or any other suitable volatile or non-volatile storage device. Persistent storage 508 may take various forms depending on the particular implementation. For example, persistent storage 508 may be a hard drive, a flash memory, a rewritable optical disk, a rewritable magnetic tape, or some combination of the above.

Communications unit 510, in these examples, provides for communications with other data processing systems or devices. In these examples, communications unit 510 is a network interface card. Communications unit 510 may provide communications through the use of either or both physical and wireless communications links. Input/output unit 512 allows for input and output of data with other devices that may be connected to data processing system 500. For example, input/output unit 512 may provide a connection for user input through a keyboard and mouse. The input/output unit may also provide access to external program code 516 stored on a computer readable media 520. In addition, input/output unit 512 may send output to a printer. Display 514 provides a mechanism to display information to a user.

Those of ordinary skill in the art will appreciate that the hardware in FIG. 5 may vary depending on the implementation. Other internal hardware or peripheral devices, such as flash memory, equivalent non-volatile memory, or optical disk drives and the like, may be used in addition to or in place of the hardware depicted in FIG. 5.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The description of the invention has been presented for purposes of illustration and description, and is not intended to be exhaustive or to limit the invention in the form disclosed. Many modifications and variations will be apparent to those of ordinary skill in the art without departing from the scope of the invention. The embodiments were chosen and described in order to explain the principles and applications of the invention, and to enable others of ordinary skill in the art to understand the invention. The invention may be implemented in various embodiments with various modifications as are suited to a particular contemplated use.

1. A computer-implemented method of managing personalized nutrition, the method comprising:
acquiring with a portable device a collection of specified physical metrics of a user;
storing the collection of specified physical metrics on the portable device;
linking, by one or more processors, the stored collection of specified physical metrics with information about the user, pre-stored on a storage device separate from the portable device, to determine nutrition for the user; and
augmenting, by the one or more processors, said linking with information obtained over the Internet to identify a place, within a defined geographical proximity to the user, to obtain said nutrition for the user.

2. The method according to claim 1, wherein:
the acquiring with a portable device a collection of specified physical metrics of a user includes using the portable device to measure a glucose level of the user; and
the linking the stored collection of specified physical metrics with information about the user includes
comparing the glucose level of the user with thresholds stored in the separate storage device; and
based on said thresholds, the portable device issuing a warning to the user and an additional preset message to additional parties through definable messaging methods.

3. The method according to claim 1, wherein:
the acquiring with a portable device a collection of specified physical metrics of a user includes acquiring a value for one of the physical metrics; and
the linking the stored collection of specified physical metrics with information about the user includes comparing said acquired value for the one of the physical metrics with a range for said one of the physical metrics, pre-stored on the separate storage device, to determine whether the acquired value for the one of the physical metrics is within said pre-stored range.

4. The method according to claim 1, wherein:
the acquiring with a portable device a collection of specified physical metrics of a user includes acquiring a value for one of the physical metrics; and
the linking the stored collection of specified physical metrics with information about the user includes comparing said acquired value for the one of the physical metrics with a threshold for said one of the physical metrics, pre-stored on the separate storage device, to determine whether the acquired value for the one of the physical metrics is above or below said pre-stored threshold.

5. The method according to claim 1, wherein the linking the stored collection of specified physical metrics with information about the user includes:
under specified conditions based on the stored collection of specified metrics and the information about the user stored of the separate storage device, sending an alert to one or more of a group pre-identified on the separate storage device.

6. The method according to claim 5, wherein the sending an alert to one or more of a group pre-identified on the separate storage device includes using one or more policies stored on the separate storage device to determine whether to send the alert.

7. The method according to claim 1, wherein the augmenting said linking with information obtained over the Internet to identify a place, within a defined geographical proximity to the user, to obtain said nutrition for the user includes:
sending a notification to the user of said place to obtain said nutrition.

8. The method according to claim 1, wherein:
a plurality of food, lifestyle and health preferences of the user are stored on the separate storage device; and
the linking the stored collection of specified physical metrics with information about the user includes using one or more of the plurality of food, lifestyle and health preferences to determine the nutrition for the user.

9. The method according to claim 8, wherein:
a plurality of thresholds and policies for the specified physical metrics of the user are stored on the separate storage device; and
the linking the stored collection of specified physical metrics with information about the user further includes using the thresholds to trigger notifications, based on said policies, to select pre-identified groups.

10. The method according to claim 1, wherein the linking the stored collection of specified physical metrics with information about the user includes:
the portable device comparing one or more of the acquired specified physical metrics with thresholds stores in the separate storage device, and the portable device reading preferences of the user and notification thresholds for the specified physical metrics from the separate storage device.

11. A computer network for managing personalized nutrition, the computer network comprising:
a portable device for acquiring and storing a collection of specified physical metrics of a user;
a storage device, separate from the portable device, for storing information about the user; and
one or more processors configured for:
linking the stored collection of specified physical metrics on the portable device with the information about the user stored on the storage device to determine nutrition for the user, and
augmenting said linking with information obtained over the Internet to identify a place, within a defined geographical proximity to the user, to obtain said nutrition for the user.

12. The computer network according to claim 11, wherein:
the portable device includes a glucose reader system to measure a glucose level of the user; and
the linking the stored collection of specified physical metrics with information about the user includes comparing the glucose level of the user with thresholds stored in the separate storage device; and
based on said thresholds, the portable device issues a warning to the user and an additional preset message to additional parties through definable messaging methods.

13. The method according to claim 11, wherein:
the linking the stored collection of specified physical metrics with information about the user includes comparing an acquired value for one of the physical metrics with a range for said one of the physical metrics, pre-stored on the separate storage device, to determine whether the acquired value for the one of the physical metrics is within said pre-stored range.

14. The computer network according to claim 11, wherein:
the separate storage device stores a plurality of food, lifestyle and health preferences of the user; and
the linking the stored collection of specified physical metrics with information about the user includes using one or more of the plurality of food, lifestyle and health preferences to determine the nutrition for the user.

15. The computer network according to claim 14, wherein:
the separate storage device stores a plurality of thresholds and policies for the specified physical metrics of the user; and
the linking the stored collection of specified physical metrics with information about the user further includes using the thresholds to trigger notifications, based on said policies, to select pre-identified groups.

16. A computer readable program storage device for managing personalized nutrition, the computer readable program storage device comprising:
one or more computer readable storage mediums having program instructions embodied therein, the program instructions executable by a computer system to cause the computer system to perform the method of:
acquiring with a portable device a collection of specified physical metrics of a user;
storing the collection of specified physical metrics on the portable device;
linking the stored collection of specified physical metrics with information about the user, pre-stored on a storage device separate from the portable device, to determine nutrition for the user; and
augmenting said linking with information obtained over the Internet to identify a place, within a defined geographical proximity to the user, to obtain said nutrition for the user.

17. The computer readable program storage device according to claim 16, wherein:
the acquiring with a portable device a collection of specified physical metrics of a user includes using the portable device to measure a glucose level of the user; and
the linking the stored collection of specified physical metrics with information about the user includes:
comparing the glucose level of the user with thresholds stored in the separate storage device; and
based on said thresholds, the portable device issuing a warning to the user and an additional preset message to additional parties through definable messaging methods.

18. The computer readable program storage device according to claim 16, wherein:
the acquiring with a portable device a collection of specified physical metrics of a user includes acquiring a value for one of the physical metrics; and
the linking the stored collection of specified physical metrics with information about the user includes comparing said acquired value for the one of the physical metrics with a range for said one of the physical metrics, pre-stored on the separate storage device, to determine whether the acquired value for the one of the physical metrics is within said pre-stored range.

19. The computer readable program storage device according to claim 16, wherein:
the acquiring with a portable device a collection of specified physical metrics of a user includes acquiring a value for one of the physical metrics; and
the linking the stored collection of specified physical metrics with information about the user includes comparing said acquired value for the one of the physical metrics with a threshold for said one of the physical metrics, pre-stored on the separate storage device, to determine whether the acquired value for the one of the physical metrics is above or below said pre-stored threshold.

20. The computer readable program storage device according to claim 16, wherein the linking the stored collection of specified physical metrics with information about the user includes:
under specified conditions based on the stored collection of specified metrics and the information about the user stored of the separate storage device, sending an alert to one or more of a group pre-identified on the separate storage device.
